(19) United States
(12) Patent Application Publication
(10) Pub. No.: US 2021/0177946 A1
(43) Pub. Date: Jun. 17, 2021

(54) THERAPEUTIC MEDICATIONS FOR THE SPHENOPALATINE GANGLION

(71) Applicant: Ira SANDERS, North Bergen, NJ (US)

(72) Inventor: Ira SANDERS, North Bergen, NJ (US)

(21) Appl. No.: 17/272,271

(22) PCT Filed: Aug. 28, 2019
(86) PCT No.: PCT/US2019/048654

§ 371 Date: Feb. 26, 2021

Related U.S. Application Data
(60) Provisional application No. 62/918,602, filed on Feb. 6, 2019.
(60) Provisional application No. 62/723,828, filed on Aug. 28, 2018.

(51) Int. Cl. A61K 38/48

(52) U.S. Cl. CPC A61K 38/4893; C12Y 304/24069

(57) ABSTRACT

Disclosed herein are methods and compositions for treatment of neurological-related disorders, and pain syndrome associated with disorders using botulinum toxin targeted to nerve ganglia.

### CROSS-REFERENCE

This application claims the benefit of U.S. Provisional Application No. 62/723,828, filed Aug. 28, 2018, and U.S. Provisional Application No. 62/918,602, filed Feb. 6, 2019, which applications are incorporated herein by reference.

### BACKGROUND OF THE INVENTION

While pain syndromes, including headache and associated pain, affect a large percentage of the population, methods for treating pain are lacking.

### SUMMARY OF THE INVENTION

Disclosed herein are methods of treating a neurological-related disorder comprising applying a therapeutically effective amount of botulinum neurotoxin to nerve ganglia including sphenopalatine ganglia and/or other ganglia of the head and neck. In some embodiments, the nerve ganglia is a parasympathetic nerve ganglia. In yet other embodiments, the nerve ganglia is a sphenopalatine ganglia, a ciliary ganglia, a submandibular ganglia, superior cervical ganglia, trigeminal ganglia and/or an otic ganglia. In still other embodiments, the nerve ganglia is a sphenopalatine ganglia. In some instances, the botulinum neurotoxin is applied to a pterygopalatine fossa. In still other instances, the botulinum neurotoxin is applied to the sphenopalatine ganglia. In still other embodiments, the botulinum neurotoxin is applied zygomatically, intranasally, through a hard palate technique, using a high tuberosity approach or combinations thereof.

In some instances, the neurological disorder is chosen from the group consisting of cluster headache, migraine headache, trigeminal neuralgia, herpes zoster pain, facial head or neck pain from various sources, complex regional pain syndrome, nasal contact point headache and vasomotor rhinitis, TMJ disorders, headaches, migraines, myofascial pain and dysfunction, anxiety, panic attacks, problems associated with Autonomic Sympathetic Overload, dizziness, vertigo, tinnitus, vomiting and nausea related to chemotherapy or other disorders, high blood pressure, atrial fibrillation increased appetite and obesity, loss of libido in women and combinations thereof. In yet other instances, the botulinum neurotoxin is chosen from the group consisting of botulinum neurotoxin type A, botulinum neurotoxin type B, botulinum neurotoxin type C, botulinum neurotoxin type D, botulinum neurotoxin type E, botulinum neurotoxin type F, botulinum neurotoxin type G, and combinations thereof. In some instances, the botulinum neurotoxin type B is administered with epinephrine. In still other instances, the botulinum neurotoxin type B further comprises a basic solution. In yet other instances, the amount of botulinum neurotoxin administered is between about 0.1 to about 1000 units. In still other instances, the amount of botulinum neurotoxin administered is between about 1 to about 1000 units. In still other embodiments, the amount of botulinum neurotoxin administered is between about 2 to about 50 units.

In some instances, the botulinum neurotoxin is administered over a period of time. In other instances, the botulinum neurotoxin is administered over one minute. In yet other instances, the volume of botulinum neurotoxin administered is between 0.1 to 10 cc. In some instances, the botulinum neurotoxin is further administered locally to the skin. In yet other instances, the botulinum neurotoxin is made from recombinant genetic methods. In still other instances, the botulinum toxin is isolated from Clostridia botulinum or Clostridia berratti.

Also disclosed herein are methods and compositions for treating a pain syndrome comprising applying a therapeutically effective amount of botulinum neurotoxin to nerve ganglia including sphenopalatine ganglia and/or other ganglia of the head and neck. In some instances, the pain syndrome is chosen from the group consisting of migraine headaches, including migraine headaches with aura, migraine headaches without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches; chronic cluster headaches; cluster variants; chronic paroxysmal hemicrania; hemicrania continua; post-traumatic headache; post-traumatic neck pain; post-herpetic neuralgia involving the head or face; pain from spine fracture secondary to osteoporosis; arthritis pain in the spine, headache related to cerebrovascular disease and stroke; headache due to vascular disorder; reflex sympathetic dystrophy, cervicalgia; glossodynia, carotidynia; cricoidynia; otalgia due to middle ear lesion; gastric pain; sciatica; maxillary neuralgia; laryngeal pain, myalgia of neck muscles; trigeminal neuralgia; post-lumbar puncture headache; low cerebro-spinal fluid pressure headache; temporomandibular joint disorder; atypical facial pain; ciliary neuralgia; paratrigeminal neuralgia; petrosal neuralgia; Eagle's syndrome; idiopathic intracranial hypertension; orofacial pain; myofascial pain syndrome involving the head, neck, and shoulder; chronic migraneous neuralgia, cervical headache; paratrigeminal paralysis; sphenopalatine ganglion neuralgia; carotidynia; Vidian neuralgia; and causalgia; trigeminal neuralgia, herpes zoster; back pain and sciatica and combinations thereof. In yet other instances, the botulinum neurotoxin is chosen from the group consisting of botulinum neurotoxin type A, botulinum neurotoxin type B, botulinum neurotoxin type C, botulinum neurotoxin type D, botulinum neurotoxin type E, botulinum neurotoxin type F, botulinum neurotoxin type G, and combinations thereof. In some instances, the botulinum neurotoxin type B is administered with epinephrine. In still other instances, the botulinum neurotoxin type B further comprises a basic solution.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

We have unexpectedly found that application of botulinum neurotoxin to the sphenopalatine ganglion, and/or one of the alternative ganglia, superior cervical ganglia, trigeminal ganglia, stellate ganglia and otic ganglia, results in improvement or positive change in the following: atrial fibrillation, angina, anorgasmic syndrome, anxiety, depression, dizziness or vertigo, esophageal spasm, hypertension, mood, libido, insomnia, myofascial pain and dysfunction, obstructive sleep apnea, glaucoma, glossodynia (commonly known as “Burning Mouth Syndrome”), nausea and vomiting, panic attacks, post-traumatic stress disorder (PTSD), tachycardia, tinnitus, TMJ disorder, vasomotor rhinitis, writer's cramp, and combinations thereof.

In addition, application of botulinum neurotoxin to the sphenopalatine ganglion, and/or one of the alternative ganglia, superior cervical ganglia, trigeminal ganglia, stellate ganglia and otic ganglia can be used to treat pain syndromes. In some embodiments, the pain syndromes include but are not limited to the following: migraine headaches, including migraine headaches with aura, migraine headaches without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches; chronic cluster headaches; cluster variants; chronic paroxysmal hemicrania; hemicrania continua; post-traumatic headache; post-traumatic neck pain; post-herpetic neuralgia involving the head or face; pain from spine fracture secondary to osteoporosis; arthritis pain in the spine, headache related to cerebrovascular disease and stroke; headache due to vascular disorder; reflex sympathetic dystrophy, cervicalgia; glossodynia, carotidynia; cricoidynia; otalgia due to middle ear lesion; gastric pain; sciatica; maxillary neuralgia; laryngeal pain, myalgia of neck muscles; trigeminal neuralgia; post-lumbar puncture headache; low cerebro-spinal fluid pressure headache; temporomandibular joint disorder; atypical facial pain; ciliary neuralgia; paratrigeminal neuralgia; petrosal neuralgia; Eagle's syndrome; idiopathic intracranial hypertension; orofacial pain; myofascial pain syndrome involving the head, neck, and shoulder; chronic migraneous neuralgia, cervical headache; paratrigeminal paralysis; sphenopalatine ganglion neuralgia; carotidynia; Vidian neuralgia; and causalgia; trigeminal neuralgia, herpes zoster; back pain and sciatica. Decreases in number or severity of migraine headache or cluster headache, facial head or neck pain from various sources, complex regional pain syndrome, and nasal contact point headache may occur with administration of a therapeutically effective amount of botulinum neurotoxin to the sphenopalatine ganglion, and/or one of the alternative ganglia, superior cervical ganglia, trigeminal ganglia, stellate ganglia and otic ganglia.

The effect of administration of a therapeutically effective amount of botulinum neurotoxin to the sphenopalatine ganglion, and/or one of the alternative ganglia, superior cervical ganglia, trigeminal ganglia, stellate ganglia and otic ganglia is completely unexpected as the mechanism of BoNT is completely different than lidocaine, cocaine and similar local anesthetics. BoNT works only at synapses whereas lidocaine works throughout a nerve, and BoNT is believed to work on cholinergic nerves, whereas local anesthetics work on all nerves: motor, sensory, parasympathetic and sympathetic.

Investigators have in the past applied botulinum neurotoxin to treat various diseases, but none have applied botulinum neurotoxin to nerve ganglia for the treatment of the disorders and diseases above.

For example, in 1994 Sanders and Shaari U.S. Pat. No. 5,766,605, Treatment of autonomic nerve dysfunction with botulinum toxin) discovered that BoNT could be used to treat disorders of the autonomic nerves. Indications included rhinorrhea, asthma, and decreasing sweating (hyperhidrosis).

Dr William Binder noted in the early 1990s that patients in whom he had injected botulinum toxin into their facial muscles to decrease wrinkles also had decreases in migraine headache. Patent U.S. Pat. No. 5,714,468 awarded to William Binder described how injections of botulinum toxin into muscles of the face or neck could alleviate migraine headaches. Since that time much research has corroborated these findings.

In 2002, Dr Ira Sanders discovered that application of BoNT to the nose or sphenopalatine ganglion (SPG) decreased all symptoms of allergic rhinitis (sneezing, itching, congestion and rhinorrhea) and asthma (U.S. Pat. Nos. 8,088,360, 9,314,513 8,092,781, 8,349,292, 7,879,340).

In 2003, Dr Ira Sanders discovered that botulinum toxin applied to the SPG could decrease migraine headaches (U.S. Pat. No. 9,504,735). More recently, Bratback et al. confirmed Dr Sanders' invention by showing that 25 units of botulinum toxin applied bilaterally to the PPF and SPG can cause a 50% decrease in migraine headaches' and a similar decrease in the number of cluster headaches.

In both studies the authors carefully chronicled all changes in the subjects yet other than headache, the improvement in other symptoms and conditions claimed in this invention were not seen.

### Botulinum Neurotoxin

Wild type clostridial neurotoxins, specifically those from Clostridia botulinum, are amphipathic protein conjugates with unique properties that make them beneficial in medical applications. First, in their natural or wild type form, they have specificity for neurons, particularly motor neurons. Second, they can block neuromuscular transmission for extended periods, from days to months depending on the serotype. Third, in most clinical applications they have been used at doses that are below the level of immunological recognition. Fourth, as they are remarkably safe for human use when injected into local areas such as muscles because there is little systemic spread of the toxin.

The clostridial neurotoxins include seven serotypes of botulinum neurotoxins, termed A-G (A, B, C1, D, E, F, and G) (Simpson, et al., Pharmacol. Rev., 33:155-188, 1981), and a single serotype of tetanus toxin (tetanus neurotoxin). These toxins all have a molecule size of ^{˜}150 kD and are comprised of a heavy-chain (^{˜}100 kD) and a light chain (^{˜}50 kD) that are covalently linked by a disulphide bridge at their N-terminals. The heavy chain consists of the binding domain (fragment C) at the C terminal and a translocation domain (fragment B, which is the amphipathic protein) at the N-terminal end. The light chain (fragment A) is the toxic domain, however, it also contains its own small amphipathic region. These neurotoxins are exceptional due to their specific binding to neurons and their specific catalytic action on the SNARE proteins, which are involved in neurotransmission. Botulinum neurotoxins A, C, and E cleave SNAP-25, in addition botulinum neurotoxin/C cleaves syntaxin 1. botulinum neurotoxins B, D, F, G and tetanus toxin cleave VAMP-2.

After a clostridial neurotoxin binds to the presynaptic surface, it is internalized by incorporation into endosomes. When the interior of the endosome reaches about pH 5.5, the amphipathic B-fragment merges with the membrane and forms a pore that allows the light chain to pass through to the cell's cytoplasm. While passing through the membrane the disulfide bond is broken and the light chain is released into the cytoplasm and exerts its toxic effect.

The toxic action of all clostridial neurotoxin light chains is to cleave proteins necessary for attachment of internal vesicles to the cell membrane. The production and docking of these vesicles is a highly regulated process that is present in all eukaryotic cells including single-cell organisms such as yeast. The vesicle membranes merge with the cell membrane thereby adding new membrane bound proteins while simultaneously discharging the vesicle's contents into the extracellular environment. In neurons, these vesicles contain neurotransmitters and neuropeptides. Botulinum neurotoxin A and E cleaves SNAP-25; botulinum neurotoxin C cleaves SNAP-25 and syntaxin 1; and tetanus neurotoxin and botulinum neurotoxin types B, D, F and G cleave VAMP (vesicle associated membrane protein, also called synaptobrevin).

There are 7 immunologically distinct toxins: A, B, C1, D, E, F and). These toxins bind to presynaptic membranes of target nerves and appear to work in a similar fashion (Brin, et al., “Report of the Therapeutics and Technology Assessment Subcommittee of the American Academy of Neurology”, Neurology, 40:1332-1336, 1990). Botulinum toxin shows a high affinity for cholinergic neurons. Botulinum toxin A produces a reversible, flaccid paralysis of mammalian skeletal muscle, presumably by blocking the exocytosis of acetylcholine at peripheral, presynaptic cholinergic receptors (Rabasseda, et al., Toxicon, 26:329-326, 1988). However, flaccid muscular paralysis is not necessary to achieve the reduction or prevention of migraine symptomatology. In fact, headache pain reduction may be observed at dosages of presynaptic neurotoxin which are lower or higher than dosages required to produce flaccid paralysis of skeletal muscle and without introduction of the neurotoxin into muscle tissue (Binder, U.S. Pat. No. 5,714,468).

Although the molecular basis for the sensation of migraine pain is not clear (Goadsby, et al., N. Eng. J. Med., 346:257-270, 2004), Botulinum toxin might exert its analgesic effect by blocking the release of nociceptive and inflammatory agents that are released during migraine, and not by blocking the release of acetylcholine. Because Botulinum toxin does not act on acetylcholine directly, but on the SNARE protein complex that mediates vesicle fusion, the release of other molecules which is also mediated by the SNARE protein complex is also affected by the toxin (Aoki, Current Medicinal Chemistry, 11:3085-3092, 2004). In fact, studies have shown that Botulinum toxin can also block the release of substance P, which is associated with neurogenic inflammation and pain generation (Aoki, Current Medicinal Chemistry, 11:3085-3092, 2004), glutamate, also associated with nociception (Cui, et al., Pain, 107:125-133, 2004), epinephrine, norepinephrine, and calcitonin gene-related peptide (Aoki, Current Medicinal Chemistry, 11:3085-3092, 2004). Botulinum toxin A does not appear to cause degeneration of nervous or muscular tissue and has been approved for use in certain therapies by the U.S. Food and Drug Administration.

In addition to Botulinum toxin A, other presynaptic neurotoxins have also been suggested to be useful for the treatment of disease, given the functional characteristics they share with Botulinum toxin (Binder, U.S. Pat. No. 5,714,468). One of these presynaptic neurotoxins is Tetanus neurotoxin, which is produced by Clostridium tetani (DasGupta, et al., Biochemie, 71:1193-1200, 1989), and shows significant sequence homology with serotypes A and E of Botulinum toxin. In particular, fragment Ibc of the Tetanus toxin, which is obtained by peptide digestion of the toxin, appears to act peripherally to produce flaccid paralysis (Fedinic, et al., Boll.lst. Sieroter Milan, 64: 35-41, 1985; and, Gawade, et al., Brain Res., 334:139-46, 1985).

### Pterygopalatine Fossa (PPF)

The pterygopalatine fossa (PPF) is an inverted pyramidal space located inferior to the orbital apex, which contains the sphenopalatine ganglion (SPG), also known as pterygopalatine ganglion, Meckel's ganglion, or Sluder's ganglion. The PPF contains various arteries, veins, lymphatics, and nerves. Preganglionic parasympathetic facial nerve fibers synapse in the PPF, while postganglionic sympathetic fibers from the superior cervical ganglion and sensory fibers from the maxillary nerve pass through the ganglion without synapsing. The PPF communicates with the orbit, nasal cavity, and oral cavity, and through the orbit with the maxillary sinus and upper teeth, which makes it an important cranial neurovascular crossroad.

In Sluder's original 1908 article, he described a variety of neuralgic, motor, sensory, and gustatory symptoms, referred to as Sluder's neuralgia, which are now called cluster headaches. Dr. Sluder was the first to propose and use transnasal injections of cocaine to anesthetize the SPG, and described using a straight needle to enter the naris, reach the PPF, push posteriorly 0.66 cm, and inject topical cocaine to bathe the ganglion. Four years later, Sluder reported that injecting a 5% solution of phenol (carbolic acid), a neurolytic substance, dissolved into alcohol instead of cocaine provided longer term pain relief from these neuralgias in 10 of his patients.

In 1925, Ruskin became the first to use SPG blocks for trigeminal neuralgia. He also introduced transoral approaches for blocking the ganglion. Since the early 1900s, the SPG has been targeted to relieve head pain; among the earliest of these treatments involved applying numbing medications on cotton swabs to the back of the nose. Another technique later used was injecting patients through an area on the cheek, using alcohol.

Byrd and Byrd described over 2,000 patients who had undergone the procedure, on whom the SPG block had been performed over 10,000 times. Despite this success and for reasons that are unclear, the use of SPG blocks dwindled and not much was published in the literature until the 1980s.

It has long been known that lidocaine, cocaine or similar local anesthetics can block the nerves in the PPF. These drugs work by numbing the membrane of the neuron and prevent transmission of impulses across thee neurons. The blocks appear to reset the autonomic nervous system turning off Sympathetic Overload (dominance) and allowing Parasympathetic response to dominate.

It has been found that application of lidocaine to the PPF or the surrounding bony canals leading to this fossa have unexpected results. Specifically, patients injected with lidocaine have improvement in cluster headache, migraine headache, trigeminal neuralgia, herpes zoster pain, facial head or neck pain from various sources, complex regional pain syndrome, nasal contact point headache and vasomotor rhinitis.

In addition, it has been found that lidocaine applied to the SPG can also improvement facial head or neck pain from various sources, complex regional pain syndrome, nasal contact point headache, mucosal headache, TMJ disorders, headaches, myofascial pain and dysfunction, anxiety, panic attacks and other problems associated with Autonomic Sympathetic Overload, vomiting and nausea related to chemotherapy or other disorders.

In addition, in patients whose SPG has been blocked there was a feeling of calmness or peace. Many patients find decreases in tension, depression, insomnia and anxiety. High blood pressure tends to stabilize and A-fib may improve. Patients may experience decreased appetite with weight loss. Women tend to have a positive effect on their libido, sometimes very soon after administration and some Anorgasmic women have reported developing normal or heightened sexual release.

Relatively recently, the FDA has approved catheters (thin plastic tube placed in the nose) to facilitate insertion of numbing medication injected in and around the SPG. Three catheters approved are Sphenocath®, Allevio®, and Tx 360®. Anesthetics used in SPG injections to control head pain include bupivacaine and lidocaine.

In a preferred embodiment, the presynaptic neurotoxin of the invention is Botulinum toxin. In a particularly preferred embodiment of the invention, the presynaptic neurotoxin is Botulinum toxin A. Botulinum toxin A is presently supplied and commercially available as “Botox”® by Allergan, Inc. of Irvine, Calif., Xeomin® of Merz Pharma of Germany, and as “Dysport”® by Ipsen, of Berkshire, UK. In another embodiment of the invention, the presynaptic neurotoxin is Botulinum toxin B. Botulinum toxin B is commercialized as “Neurobloc”®/“Myobloc”® by Solstice Neuroscience, Inc, of San Francisco, Calif. Botox® has been FDA approved to treat, among other things, cervical dystonia, brow furrows, blepharospasm, strabismus, and hyperhidrosis.

The potency of a toxin is expressed as a multiple of the LD50 value for a reference mammal. One “unit” of toxin is the amount of toxin that kills 50% of a group of mammals that were disease-free prior to inoculation with the toxin. For example, one unit of Botulinum toxin is defined as the LD50 upon intra peritoneal injection into female Swiss Webster mice weighing 18-20 grams each. One nanogram of the commercially available Botulinum toxin A typically contains about 40 mouse units. The potency in humans of the Botulinum toxin A product currently supplied by Allergan, Inc. as “Botox”® is estimated to be about LD50=2,730 units.

Assuming an approximate potency of LD50=2,730 units, the presynaptic neurotoxin can be administered in a dose of up to about 1,000 units; however, dosages of as low as about 1 unit will have therapeutic efficacy. It is important to note that the potency of a single unit is variable among the commercial formulations. The potency of 1 U of onabotulinumtoxinA (Botox) is about equal to 1 U of incobotulinumtoxinA (Xeomin), 3 U of abobotulinumtoxinA (Dysport) and 40 to 50 U of rimabotulinumtoxinB (Neurobloc). However, it is very important to recognize that this ratio of equivalence cannot be employed. For injections, botulinum toxins type A are diluted with 0.9% sodium chloride solution.

The injections will be repeated as necessary. As a general guideline, Botulinum toxin A administered into or near muscle tissue has been observed to produce flaccid paralysis at target site muscles for up to about 3 to 6 months. However, increased duration up to 9-12 months have been reported with autonomic applications such as hyperhidrosis.

In a preferred embodiment of the invention, commercially available Botox® can be reconstituted with sterile non-preserved saline prior to injection. Each vial of Botox® contains about 100 units of clostridium Botulinum toxin type A purified neurotoxin complex. Dilutions will vary depending upon the commercial preparation.

The preferred target administration sites for the current invention are the contents of the PPF including but not limited to the SPG, maxilllary nerve and sympathetic neurons.

For example, in one embodiment 100 units of Botulinum toxin A are reconstituted in 4 cc of normal saline, a specific syringe with demarcations at 0.1 cc of 2.5 units, and at 0.2 cc of 5 units will be used. In another embodiment in which 100 units of Botulinum toxin A are reconstituted in 1 cc of normal saline, the syringe with demarcations at 0.1 cc of 10 units; and at 0.2 cc of 20 units will be used. These delivery tools allow for accurate delivery and recording of the dose given.

Injections are preferably made every 3-12 months or upon return of symptoms. The dose injected on one side can vary from about 0.1-1000 units, preferably about 5-50 units, and more preferably about 20-30 units for botulinum neurotoxin type A, for example onabotulinumtoxin A (Botox). In some instances and depending upon the type of botulinum neurotoxin used, the amount employed would increase, for example, for rimbotulinumtoxin B (Neurobloc) the amount administered would be approximately 50-times the amount of botulinum neurotoxin relative to onabotulinum toxin A (botox), i.e., from about 5-50,000 units, preferably about 250-2500 units, and more preferably about 1000-1500 units for botulinum neurotoxin type B. Injections can be unilateral or bilateral depending on the nature and location of the lesion. Injections can be done simultaneously on both sides or separately.

In another embodiment of this technique, an injection of epinephrine can precede, or be given simultaneous or even after the BoNT injection. The epinephrine contracts blood vessels, thereby decreasing the soft tissue volume within the pterygopalatine fossa. It also aids in preventing or minimizing any bleeding in the fossa. In addition, it decreases the extracellular space and fluid exchange in the area, decreasing the spread of the toxin.

In another embodiment the local and systemic spread of toxin can be minimize, a non-limiting example being serotype B a used. Type B is marketed in liquid form with preservation aided by an acidic pH of 5.6. This particular product is known for its pain on injection and for seemingly substantial systemic spread to autonomically innervated structures, a non-limiting example being the salivary glands which decreases salivation which the patient experiences as dry mouth. To combat these side effects, the type B BoNT solution may be combined with epinephrine described above. In the particular case of BoNT the acidic nature of the solution may cause inflammation with increased fluid release and uptake by local blood vessels. This increased fluid exchange could remove BoNT type B from the area and allow it to enter the systemic circulation. To minimize this, a basic solution to neutralize the acid pH and decrease the acidic nature of the injection. This decreases pain and the inflammation in the area.

The application of BoNT by needle injection can be done in at least four ways.

### Zygomatic

In the suprazygomatic approach the patient is placed supine with the head in a neutral position. The needle entry point is found at the angle formed by the superior edge of the zygomatic arch below and the posterior orbital rim forward. The needle (22 to 25 gauge) is inserted perpendicular to the skin and advanced to reach the greater wing of the sphenoid at a depth of approximately 10-15 mm). The needle is then reoriented in a caudal and posterior direction and advanced a further 35-45 mm to reach the pterygopalatine fossa. After a negative aspiration test for blood, solution is slowly injected. Nerve stimulation may help locate the pterygopalatine fossa: Nerve stimulation is associated with paresthesia coinciding with the stimulating frequency of the nerve stimulator. In anesthetized children, stimulation of the temporal muscle that results in a mandibular contraction may be noted. The disappearance of the muscle contraction heralds the passage through the temporal muscle and entrance into the pterygomaxillary fossa.

In an alternative embodiment the needle can be inserted below the zygomatic arch (infrazygomatic).

### Intransal

Preferably the nose is decongested with phenylephrine or epinephrine and anesthetized with lidocaine. A flexible or rigid scope is passed into the nasal cavity such that it visualizes the mucosa posterior to the middle turbinate. A syringe contained BoNT diluted with preferably 1-4 cc of normal saline is attached to a long needle preferably about 3.5 cm and preferably 27 gauge. The needle is introduced into the nasal cavity and advanced until the needle tip is at the posterior border of the middle turbinate. The needle is then slowly advanced 1-10 mm through mucosa behind the middle turbinate. The needle is aspirated and then injection is made.

### Palatal

The exit of the pterygopalatine canal is identified on the hard palate about mid-way between the 2^{nd} or 3^{rd} molar and the midline. A needle is advanced into the foramen in a posterosuperior direction at an angle of 45-60 degrees from the horizontal plane of the hard palate. At 20-30 mm the needle is aspirated and then injection is made.

### High Tuberosity

A 25-gauge long needle is recommended for this injection but a 27-gauge is acceptable.¹ The penetration site for the maxillary block is the height of the mucobuccal fold distal to the maxillary second molar. Prior to placing topical anesthetic, it is important to use a finger to feel along the facial aspect of the maxilla to find the zygomatic process, which is usually located above the first maxillary molar. It is important to insert distal to the zygomatic process or the maxillary bone may be scraped during administration. The angle of the syringe should be 45° from the mid-sagittal plane, as well as 45° apically from the maxillary occlusal plane. A helpful visual guide for this angle is a line running from the lateral periphery of the ala of the nose to the inside corner of the opposite eyebrow. The average depth of penetration for the maxillary block is 30 mm. With a 32 mm long needle, 2 mm of needle should remain visible outside the tissue. The bone should not be contacted on this injection, and the needle should progress smoothly through the tissues. The clinician should know the exact length of the needle, as different manufacturers produce different needle lengths. If both aspirations are negative, the injection anesthetic should be slowly deposited, re-aspirating every ¼ of the cartridge to make sure a blood vessel has not been penetrated. The clinician should administer this injection slowly (taking more than 60 seconds to deliver the full amount) because of the highly vascular nature of the pterygopalatine fossa.

Injections are preferably made every 3-9 months or upon return of symptoms.

The dose injected on one side can vary from 1-1000 units, preferably 1-50 units, and more preferably 20-30 units. Injections can be unilateral or bilateral.

It is another embodiment of this invention to apply BoNT topically to the SPG. The SPG can be anesthetized by topical application of local anesthetics to the mucosa of the nasal cavity. This is done in the region immediately posterior to posterior end of the middle turbinate (termed the SPG area). This is because the SPG underlies the mucosa in this area. Moreover, topical diffusion is possible as there is no bone underneath the mucosa in this region. Moreover, local anesthetics are small molecules which are known to pass through mucosa easily. In contrast, BoNT is a large molecule that passes through mucosa poorly and diffuses slowly. However, in 1994, Sanders and Shaari showed that botulinum toxin could pass through nasal mucosa to block neurons suppling nasal secretory glands.

In one embodiment, BoNT is applied to the SPG area by contact with a cotton tipped applicator. This is like techniques used to apply local anesthetics to this area. The cotton is saturated with a solution of BoNT. It is held against the mucosa for 1 minute to 1 hour. The BoNT solutions are more concentrated or have higher doses than injection as topical delivery is less efficient and much of the solution never leaves the cotton. Dosage is preferably 1 to 1000 units dissolved in from 0.1 to 10 cc, more preferably 5 to 100 units dissolved in 0.5 to 5 cc.

A practitioner familiar with the art would know of obvious variations of this topical technique including other physical carriers (sponges) and other materials.

### EXAMPLES

### Example 1. Migraine

A 50-year-old female has 10-15 episodes of migraine headache every month. Her physician uses 1 cc of normal saline in a 5 cc syringe to dilute a 100 unit vial of BoNT. The physician injects via the zygomatic technique and deposits 0.25 cc into the PPF. Over the next month the patient's frequency of headache decreases to 5 per month and they are of lesser intensity.

### Example 2. Cluster Headache

A 40-year-old male has 5 episodes of cluster headache every month. His physician uses 2 cc of normal saline in a 5 cc syringe to dilute a 100 unit vial of BoNT. The physician injects via the palatal technique and deposits 0.5 cc into the PPF. Over the next month the patient's frequency of headache decreases to 1 per month and they are of lesser intensity.

### Example 3. Dizziness, Vertigo

A patient experiences acute onset of vertigo 2 days after noting an upper respiratory tract infection. The physician dilutes a 100 unit vial of BoNT with 4 cc of normal saline in a 5 cc syringe attached to a 3.5 inch 27 gauge spinal needle. The physician inserts the needle into one nostril via the intranasal technique and deposits 1 cc into the PPF. Over the next week the patient experiences only slight improvement in symptoms so the physician performs an intra-nasal injection on the opposite side. Within a week after the second injection the vertigo is eliminated and the dizziness decreases to a lesser intensity.

### Example 4. Tinnitus

A patient experiences chronic tinnitus which keeps him awake at night. An audiogram shows bilateral high frequency sensorineural hearing loss. The physician dilutes a 100 unit vial of BoNT with 2 cc of normal saline in a 5 cc syringe with a 1.5 inch 27 gauge needle. The physician injects 0.5 cc in the PPF bilaterally. The patient notes decreased tinnitus over the next week and resolution of his insomnia which lasts 6 months.

### Example 5. Insomnia

A patient experiences chronic tinnitus which keeps him awake at night. An audiogram shows bilateral high frequency sensorineural hearing loss. The physician dilutes a 100-unit vial of BoNT with 2 cc of normal saline in a 5 cc syringe with a 1.5 inch 27 gauge needle. The physician injects 0.5 cc in the PPF bilaterally. The patient notes decreased insomnia over the next week which lasts 9 months.

### Example 6. Decreased Libido

A 30-year-old post-menopausal female has noted decreased libido over the past year. The physician dilutes a 100 unit vial of BoNT with 4 cc of normal saline in a 5 cc syringe attached to a 3.5 inch 27 gauge spinal needle. The physician inserts the needle into one nostril via the intranasal technique and deposits 0.5 cc into the PPF. The injection is repeated on the opposite side. Over the next month the patient reports increased libido which lasts six months.

### Example 7. Anorgasmic Syndrome

A 50-year-old post-menopausal female has noted decreased the inability to achieve orgasm despite adequate stimulation over the past year. The physician dilutes a 100 unit vial of BoNT with 4 cc of normal saline in a 5 cc syringe attached to a 3.5 inch 27 gauge spinal needle. The physician inserts the needle into one nostril via the intranasal technique and deposits 0.5 cc into the PPF. The injection is repeated on the opposite side. Over the next month the patient reports orgasm during sexual activity. This improvement lasts 5 months.

### Example 8. Trigeminal Neuralgia

A 30-year-old male has lancinating pain around the right side of his face while chewing. He is diagnosed with trigeminal neuralgia. His physician uses 2 cc of normal saline in a 5 cc syringe to dilute a 100 unit vial of BoNT. The physician injects via the palatal technique and deposits 0.5 cc into the PPF unilaterally on the side of the pain. Over the next month the patient's frequency of pain decreases 80% and this lasts 12 months.

### Example 9. Temporomandibular Joint (TMJ) Pain

A 40-year-old female has pain localized to her left ear when chewing. Her dentist diagnoses TMJ. The dentist dilutes a 100-unit vial of BoNT with 2 cc of normal saline in a 5-cc syringe with a 1.5 inch 27 gauge needle. He anesthetizes the mucosa and then performs a high tuberosity injection of 1 cc on the left side. Over 2 weeks thee TMJ pain diminishes and the effect lasts 8 months. 10. Hypertension. A 60 ear male has hypertension with a blood pressure reading of 140/90. The physician dilutes a 100 unit vial of BoNT with 4 cc of normal saline in a 5 cc syringe attached to a 3.5 inch 27 gauge spinal needle. The physician inserts the needle into one nostril via the intranasal technique and deposits .5 cc into the PPF. The injection is repeated on the opposite side. In 2 weeks his blood pressure reading is 120/70 and is no longer hypertensive. The effect lasts 3 months.

### Example 11. Atrial Fibrillation

A 70 ear male has atrial fibrillation. The physician dilutes a 100 unit vial of BoNT with 4 cc of normal saline in a 5 cc syringe attached to a 3.5 inch 27 gauge spinal needle. The physician inserts the needle into one nostril via the intranasal technique and deposits 0.5 cc into the PPF. The injection is repeated on the opposite side. In 2 weeks his atrial fibrillation has been eliminated. The effect lasts 6 months.

### Example 12. Anxiety and Depression

A 60 year old female suffers from anxiety and depression. The physician dilutes a 100 unit vial of BoNT with 4 cc of normal saline in a 5 cc syringe attached to a 3.5 inch 27 gauge spinal needle. The physician inserts the needle into one nostril via the intranasal technique and deposits 0.5 cc into the PPF. The injection is repeated on the opposite side. Over the next month the patient reports that the anxiety and depression is largely eliminated. This improvement lasts 5 months.

### Example 13. Panic Attacks

A 40-year-old female military veteran has anxiety and panic attacks. His physician uses 2 cc of normal saline in a 5-cc syringe to dilute a 100 unit vial of BoNT. The physician injects via the palatal technique and deposits 0.5 cc into the PPF bilaterally. Over the next 2 months the number and severity thee panic attacks decreases 80% and this lasts 12 months.

### Example 14. Post Traumatic Stress Disorder (PTSD)

A 30-year-old male military veteran has PTSD. His physician uses 2 cc of normal saline in a 5-cc syringe to dilute a 100 unit vial of BoNT. The physician injects via the palatal technique and deposits 0.5 cc into the PPF bilaterally. Over the next 2 months the patient's severity of PTSD decreases 80% and this lasts 12 months.

### Example 15. Vomiting and Nausea

A 40-year-old female has cancer and is undergoing chemotherapy. After each dose of chemotherapy, she experiences 1 week of nausea and vomiting. The physician dilutes a 100-unit vial of BoNT with 4 cc of normal saline in a 5 cc syringe attached to a 3.5 inch 27 gauge spinal needle. The physician inserts the needle into one nostril via the intranasal technique and deposits 0.5 cc into the PPF. The injection is repeated on the opposite side. Over the next month the patient reports decreased nausea and vomiting which lasts six months.

### Example 16. Glaucoma

A 30-year-old male has been diagnosed with glaucoma. His physician uses 2 cc of normal saline in a 5-cc syringe to dilute a 100-unit vial of BoNT. The physician injects via the palatal technique and deposits 0.5 cc into the PPF bilaterally. Over the next month the patient's frequency of pain decreases 80% and this lasts 12 months.

### Example 17. Maxillary Cancer

A 60-year-old male has cancer involving his right maxillary sinus with severe continual pain and inability to swallow. His physician dilutes 2 100-unit vial of BoNT with 1 cc of normal saline each and places them in a 5-cc syringe with a 1.5 inch 27 gauge needle. He anesthetizes the mucosa and then performs a high tuberosity injection of 1 cc on the left side. Over 3 days the pain diminishes to a tolerable level.

### Example 18. Herpes Zoster

A 40-year-old male has herpes zoster of the face affecting his rights eye with severe pain. The nasal cavity is anesthetized with lidocaine 1% with epinephrine 1:100,000. A cotton tipped applicator is saturated with 1 cc of a solution of normal saline containing 100 units of BoNT. The applicator is introduced into the nasal cavity and the cotton is held against the SPG area mucosa for 1 hour. In 2 days, the pain of the herpes zoster has decreased by 50%.

Sluder G. The role of the sphenopalatine ganglion in nasal headaches. N Y State J Med. 1908; 27:8-13.

Byrd H, Byrd W. Sphenopalatine phenomena: present status of knowledge. Arch Intern Med. 1930; 46:1026-1038.

Maizels, M; Scott B; Cohen W; Chen W. Intranasal lidocaine for treatment of migraine: a randomized, double blind, controlled trial. JAMA 1996; 27:319-21.

Piagkou, M; Demesticha, T; Troupis, T; Vlasis, K; Skandalakis, P, Makri, A; Mazarakis, A; Lappas, D; Piagkos, G; Johnson, E O. “The pterygopalatine ganglion and its role in various pain syndromes: from anatomy to clinical practice.” Pain Pract.; 2012; 12(5):399-412.

Jenkins B; Tepper, S J. “Neurostimulation for Primary Headache Disorders, Part 1: Pathophysiology and Anatomy, History of Neuromodulation in Headache Treatment, and Review of Peripheral Neuromodulation in Primary Headaches.” Headache 2011; 51:1254-1266.

Martelletti, P; Jensen, R H; Antal, A; Arcioni, R; Brighina, F'de Tommaso, M; Franzini, A; Fontaine, D; Heiland, M; Jürgens, T P; Leone, M; Magis, D; Paemeleire, K; Palmisani, S; Paulus, W; May, A. “Neuromodulation of chronic headaches: position statement from the European Headache Federation.” J Headache Pain 2 013; 14(1):86.

Khan, S; Schoenen, J; Ashina, M. “Sphenopalatine ganglion neuromodulation in migraine: What is the rationale?” Cephalalgia 2014; 34(5:382-391.

Schoenen, J; Jensen, R H; Lantéri-Minet, M; Láinez, M J; Gaul, C; Goodman, A M; Caparso, A; May, A. “Stimulation of the sphenopalatine ganglion (SPG) for cluster headache treatment. Pathway CH-1: a randomized, sham-controlled study.” Cephalalgia. 2013 July; 33(10):816-30.

Lee, S. H. Efficacy of sphenopalatine ganglion block in nasal mucosal headache presenting as facial pain. Cranio. 2018 Jun. 8:1-3. doi: 10.1080/08869634.2018.1475859. [Epub ahead of print]

https://www.sphenopalatineganglionblocks.com/.

Bratbak D F, Nordgård S, Stovner L J, Linde M, Dodick D W, Aschehoug, Folvik, Tronvik E. Pilot study of sphenopalatine injection of onabotulinumtoxinA for the treatment of intractable chronic migraine. Cephalalgia. 2017 April; 37(4):356-364. doi: 10.1177/0333102416648328. Epub 2016 May 6.

Bratbak D F1, Nordgard S2, Stovner L J3, Linde M4, Folvik M5, Bugten V2, Tronvik E4. Cephalalgia. Pilot study of sphenopalatine injection of onabotulinumtoxinA for the treatment of intractable chronic cluster headache. 2016 May; 36(6):503-9. doi: 10.1177/0333102415597891. Epub 2015 Jul. 31.

Malamed S. Handbook of Local Anesthesia. 6th ed. St. Louis: Elsevier Mosby; 2013.

Maizels, M; Scott B; Cohen W; Chen W. Intranasal lidocaine for treatment of migraine: a randomized, double blind, controlled trial. JAMA 1996; 27:319-21.

Piagkou, M; Demesticha, T; Troupis, T; Vlasis, K; Skandalakis, P; Makri, A; Mazarakis, A; Lappas, D; Piagkos, G; Johnson, E O. “The pterygopalatine ganglion and its role in various pain syndromes: from anatomy to clinical practice.” Pain Pract.; 2012; 12(5):399-412.

Jenkins B; Tepper, S J. “Neurostimulation for Primary Headache Disorders, Part 1: Pathophysiology and Anatomy, History of Neuromodulation in Headache Treatment, and Review of Peripheral Neuromodulation in Primary Headaches.” Headache 2011; 51:1254-1266.

Martelletti, P; Jensen, R H; Antal, A; Arcioni, R; Brighina, F'de Tommaso, M; Franzini, A; Fontaine, D; Heiland, M; Jiirgens, T P; Leone, M; Magis, D; Paemeleire, K; Palmisani, S; Paulus, W; May, A. “Neuromodulation of chronic headaches: position statement from the European Headache Federation.” J Headache Pain 2 013; 14(1):86.

Khan, S; Schoenen, J; Ashina, M. “Sphenopalatine ganglion neuromodulation in migraine: What is the rationale?” Cephalalgia 2014; 34(5:382-391.

Schoenen, J; Jensen, R H; Lantéri-Minet, M; Láinez, M J; Gaul, C; Goodman, A M; Caparso, A; May, A. “Stimulation of the sphenopalatine ganglion (SPG) for cluster headache treatment. Pathway CH-1: a randomized, sham-controlled study.” Cephalalgia. 2013 July; 33(10):816-30.

U.S. Pat. No. 4,921,757A—1985 Apr. 26 1990 May 1 Massachusetts Institute of Technology, System for delayed and pulsed release of biologically active substances.

WO1995017904A2—1993 Dec. 28 1995 Jul. 6 Allergan, Botulinum toxins for treating various disorders and associated pain.

WO1995028171A1—1994 Apr. 15 1995 Oct. 26 Mount Sinai School of Medicine of The City University of New York, Treatment of autonomic nerve dysfunction with botulinum toxin.

U.S. Pat. No. 5,670,484A—1994 May 9 1997 Sep. 23 Binder; William J., Method for treatment of skin lesions associated with cutaneous cell-proliferative disorders.

WO2000013599A1—1998 Sep. 9 2000 May 16 Vida Derm, Systems and methods for ablating discrete motor nerve regions.

U.S. Pat. No. 6,063,768A—1997 Sep. 4 2000 May 16 First; Eric R., Application of botulinum toxin to the management of neurogenic inflammatory disorders.

WO2001026674A2—1999 Oct. 13 2001 Apr. 19 Allergan Sales, Inc., Method for treating otic disorders.

WO2001026736A2—1999 Oct. 12 2001 Apr. 19 Allergan Sales, Inc., Intraspinal compositions containing botulinum toxin for treating pain.

U.S. Pat. No. 6,299,893B1—2000 Apr. 17 2001 Oct. 9 Marvin Schwartz, Method to reduce hair loss and stimulate hair regrowth.

WO2002040506A2—2000 Nov. 17 2002 May 23 Allergan Inc., Modified clostridial neurotoxins with altered biological persistence.

US20020102274A1—2000 Feb. 15 2002 Aug. 1 Allergan Sales, Inc., Clostridial toxin therapy for Hashimoto's thyroiditis.

U.S. Pat. No. 6,429,189B1—1999 Dec. 10 2002 Aug. 6 Botulinum Toxin Research Associates, Inc. Cytotoxin (non-neurotoxin) for the treatment of human headache disorders and inflammatory diseases.

U.S. Pat. No. 6,458,365B1—1993 Dec. 28 2002 Oct. 1 Allergan, Inc., Method for treating headache.

U.S. Pat. No. 6,464,986B1—2000 Apr. 14 2002 Oct. 15 Allegan Sales, Inc., Method for treating pain by peripheral administration of a neurotoxin.

US20020192239A1—2001 Jan. 9 2002 Dec. 19 Borodic Gary E., Use of botulinum toxin for the treatment of chronic facial pain.

U.S. Pat. No. 6,500,436B2—2000 Jan. 19 2002 Dec. 31 Allergan, Inc., Clostridial toxin derivatives and methods for treating pain.

U.S. Pat. No. 6,565,870B1—2000 Apr. 28 2003 May 20 Allergan, Inc., Methods for treating bone tumors.

US20030100574A1—2001 Nov. 15 2003 May 29 Wilson Nestor Antonio Lagos, Use and application of a pharmaceutical composition containing a mixture of natural-origin heterocyclical guanidine, for cosmetology, wound healing, focal dystonia and muscular spasm-related clinical pathologies.

US20030118598A1—2000 Feb. 8 2003 Jun. 26, Allergan, Inc. Clostridial toxin pharmaceutical compositions.

US20030138437A1—2000 Feb. 8 2003 Jul. 24 Allergan, Inc., Reduced toxicity clostridial toxin pharmaceutical compositions.

WO2002000172A3—2000 Jun. 28 2003 Aug. 7 Ira Sanders, Methods for using tetanus toxin for beneficial purposes in animals (mammals).

U.S. Pat. No. 6,623,742B2—2001 Sep. 17 2003 Sep. 23 Allergan, Inc., Methods for treating fibromyalgia.

U.S. Pat. No. 6,641,820B1 2000 Jan. 19 2003 Nov. 4 Allergan, Inc., Clostridial toxin derivatives and methods to treat pain.

US20030208042A1—2000 Feb. 24 2003 Nov. 6 Brown University Research Foundation, Alpha-bungarotoxin molecules and uses thereof.

US20030211121A1—2002 May 10 2003 Nov. 13 Allergan Sales, Inc., Therapeutic treatments for neuropsychiatric disorders.

US20030224019A1—2002 Mar. 1 2003 Dec. 4 O'Brien Christopher, Methods of treating nerve entrapment syndromes.

US20040014663A1—1993 Dec. 28 2004 Jan. 22 Aoki Kei Roger, Botulinum toxin treatment for cervical dystonia.

U.S. Pat. No. 6,688,311B2 2002 Mar. 14 2004 Feb. 10 Allergan, Inc., Method for determining effect of a clostridial toxin upon a muscle.

US20040086532A1—2002 Nov. 5 2004 May 6 Allergan, Inc., Botulinum toxin formulations for oral administration.

US20040086531A1—2002 Nov. 5 2004 May 6 Allergan, Inc., Methods for treating ulcers and gastroesophageal reflux disease.

US20040126397A1—1993 Dec. 28 2004 Jul. 1 Allergan, Inc., Use of the neurotoxic component of a botulinum toxin for treating various disorders and conditions and associated pain.

US20040126396A1—1993 Dec. 28 2004 Jul. 1 Allergan, Inc., Botulinum toxin treatment for strabismus.

US20040170665A1—2000 Jun. 2 2004 Sep. 2 Allergan, Inc., Intravitreal botulinum toxin implant.

US20040175390A1—2003 Mar. 6 2004 Sep. 9 Gary Borodic, Selection of patients with increased responsiveness to botulinum toxin.

US20040175399A1—2003 Mar. 3 2004 Sep. 9 Allergan, Inc., Methods for treating uterine disorders.

WO2004084839A2—2003 Mar. 24 2004 Oct. 7 Cady Roger K, Method and article for treatment of sensory neuron related disorders through transdermal application of botulinum toxin.

US20040210200A1—2003 Apr. 16 2004 Oct. 21 Gerondale Scott J., Controlled volume injection/aspiration device.

US20040213812A1—2003 Apr. 25 2004 Oct. 28 Allergan, Inc., Methods for treating trichotillomania.

US20040213813A1—2003 Apr. 25 2004 Oct. 28 Allergan, Inc., Therapy for obsessive compulsive head banging.

US20040213814A1—2003 Apr. 25 2004 Oct. 28 Allergan, Inc., Therapeutic treatments for repetitive hand washing.

US20040213815A1—2003 Apr. 25 2004 Oct. 28 Allergan, Inc., Clostridial toxin treatment for dermatillomania.

US20040213811A1—2003 Apr. 25 2004 Oct. 28, Allergan, Inc., Use of a botulinum neurotoxin to alleviate various disorders.

US20040220386A1—2000 Jul. 21 2004 Nov. 4 Steward Lance E., Clostridial neurotoxin compositions and modified clostridial neurotoxins

US20040219619A1—2000 Jul. 21 2004 Nov. 4 Ester Fernandez-Salas, Methods of identifying compounds that alter toxin persistence and/or protease activity

WO2004098714A1—2003 May 2 2004 Nov. 18 Allergan, Inc., Methods for treating sinus headache

US20040234532A1—2003 May 20 2004 Nov. 25 Allergan, Inc., Methods and compositions for treating eye disorders

US20040247606A1—2003 Mar. 6 2004 Dec. 9 Gary Borodic, Treatment of sinusitis related chronic facial pain and headache with botulinum toxin injections

US20040253274A1—2003 Jun. 11 2004 Dec. 16 Allergan, Inc., Use of a clostridial toxin to reduce appetite

US20050013850A1—2003 Jul. 15 2005 Jan. 20 Caers Jan K., Device to assist hyperhydrosis therapy

US20050031648A1—1999 Dec. 7 2005 Feb. 10 Allergan, Inc., Methods for treating diverse cancers

US20050054827A1—1998 Oct. 2 2005 Mar. 10 Lewis Richard James, Novel peptides

US20050100562A1—2001 Apr. 24 2005 May 12 Kazunori Kurita, Granular compositions and process for producing the same

US20050106182A1—2003 Nov. 17 2005 May 19 Shengwen Li, Rescue agents for treating botulinum toxin intoxications

U.S. Pat. No. 6,903,187B1 2000 Jul. 21 2005 Jun. 7 Allergan, Inc., Leucine-based motif and clostridial neurotoxins

US20050123567A1—2003 Dec. 9 2005 Jun. 9 First Eric R., Botulinum toxin therapy for skin disorders

US20050129677A1—2003 Dec. 10 2005 Jun. 16 Shengwen Li, Lipid rafts and clostridial toxins

US20050147626A1—2003 Oct. 29 2005 Jul. 7 Allergan, Inc., Botulinum toxin treatments of neurological and neuropsychiatric disorders

US20050147625A1—2004 Jan. 6 2005 Jul. 7 Allergan, Inc., Botulinum toxin treatment for kinesia

US20050148935A1—2003 Dec. 29 2005 Jul. 7 Rozalina Dimitrova, Botulinum toxin injection guide

US20050152923A1—2004 Jan. 8 2005 Jul. 14 Brin Mitchell F., Methods for treating vascular disorders

US20050191320A1—2004 Feb. 26 2005 Sep. 1 Turkel Catherine C., Methods for treating pain and for treating a medication overuse disorder

US20050191321A1—2004 Feb. 26 2005 Sep. 1 Allergan, Inc., Methods for treating headache

US20050220734A1—2004 Apr. 2 2005 Oct. 6 Allergan, Inc., Therapy for melanin related afflictions

US20050220821A1—2004 Mar. 31 2005 Oct. 6 Allergan, Inc., Pressure sore treatment

US20050241652A1—2002 Mar. 14 2005 Nov. 3 Allergan, Inc., Surface topography method for determining effects of a botulinum toxin upon a muscle and for comparing botulinum toxins

US20050287175A1—2004 Jun. 29 2005 Dec. 29 Shengwen Li, Methods of modulating intracellular degradation rates of toxins

US20060018844A1—2002 Oct. 15 2006 Jan. 26 Allergan, Inc., Botulinum toxin dental therapy for angular cheilosis

US20060024331A1—2004 Aug. 2 2006 Feb. 2 Ester Fernandez-Salas, Toxin compounds with enhanced membrane translocation characteristics

US20060024794A1—2004 Jul. 30 2006 Feb. 2 Shengwen Li, Novel methods for production of di-chain botulinum toxin

US20060039929A1—2000 Jul. 21 2006 Feb. 23 Ester Fernandez-Salas, Chimera botulinum toxin type E

US20060051377A1—2004 Sep. 3 2006 Mar. 9 Allergan, Inc., Stretch mark treatment

US20060051341A1—2004 Sep. 3 2006 Mar. 9 Allergan, Inc., Methods for treating a buttock deformity

US20060073208A1—2004 Oct. 1 2006 Apr. 6 Allergan, Inc., Cosmetic neurotoxin compositions and methods

US20060078571A1—2002 Feb. 25 2006 Apr. 13 Allergan, Inc., Methods for treating inflammation pain

US20060083758A1—2004 Oct. 20 2006 Apr. 20 Allergan, Inc., Treatment of premenstrual disorders

US20060093625A1—2000 Jan. 19 2006 May 4 Allergan, Inc., Clostridial toxin derivatives and methods for treating pain

US20060104994A1—2000 Feb. 8 2006 May 18 Hunt Terrence J, Clostridial toxin pharmaceutical composition containing a gelatin fragment

US20060171963A1—2005 Feb. 1 2006 Aug. 3 Blumenfeld Andrew M, Targeted delivery of botulinum toxin for the treatment and prevention of trigeminal autonomic cephalgias, migraine and vascular conditions

US20060182767A1—2002 May 28 2006 Aug. 17 Borodic Gary E, High-potency botulinum toxin formulations

US20060220266A1—2005 Apr. 4 2006 Oct. 5 Jean-Louis Pessin, Circulating fluid system for powder fluidization and method of performing same

US20060258843A1—2000 Nov. 17 2006 Nov. 16 Steward Lance E, Post-translational modifications and Clostridial neurotoxins

WO2006122123A2—2005 May 9 2006 Nov. 16 Levin Bruce H, Methods of alleviating disorders and their associated pain

US20060269575A1—2000 Feb. 8 2006 Nov. 30 Allergan, Inc., Botulinum toxin pharmaceutical compositions formulated with recombinant albumin

US20060269573A1—2005 May 26 2006 Nov. 30 Allergan, Inc., Methods for treating peritoneal adhesions

US20060286127A1—2005 Jun. 17 2006 Dec. 21 Allergan, Inc., Treatment of autoimmune disorder with a neurotoxin

KR100670479B1 1999 Oct. 12 2007 Jan. 18, Method For Treating Pain

US20070020295A1—2000 Jun. 2 2007 Jan. 25 Allergan, Inc., Controlled release neurotoxin system and method

US20070048334A1—2005 Aug. 24 2007 Mar. 1 Allergan, Inc., Use of a botulinum toxin to improve gastric emptying and/or to treat GERD

US20070077259A1—2005 Mar. 3 2007 Apr. 5 Revance Therapeutics, Inc., Compositions and methods for topical application and transdermal delivery of botulinum toxins

U.S. Pat. No. 7,211,261B1 1998 Sep. 11 2007 May 1 Solstice Neurosciences, Inc., Stable liquid formulations of botulinum toxin

US20070160633A1—2006 Jan. 12 2007 Jul. 12 Allergan, Inc., Methods for enhancing therapeutic effects of a neurotoxin

US20070178121A1—2006 Jan. 27 2007 Aug. 2 Allergan, Inc., Methods for enhancing skin treatments

U.S. Pat. No. 7,255,866B2 2001 Sep. 17 2007 Aug. 14 Allergan, Inc., Botulinum toxin therapy for fibromyalgia

US20070259000A1—2006 May 2 2007 Nov. 8 Allergan, Inc., Methods for alleviating testicular pain

US20070258900A1—2004 Jul. 20 2007 Nov. 8 Joseph Francis, Blood Flow Assessment of Clostridial Toxin Applications

US20070280970A1—2004 May 7 2007 Dec. 6 Phytotox Limited, Methods of Treating Wounds With Gonyautoxins

US20070286337A1—2006 May 19 2007 Dec. 13 Xuewu Wang, Detector array and device using the same

US20080021437A1—2006 Apr. 27 2008 Jan. 24 Boyd James P, Enhancement of the efficacy of a clenching reduction device by augmenting with botulinum toxin type A

US20080038203A1—2004 Mar. 3 2008 Feb. 14 Revance Therapeutics, Inc., Compositions and Methods for Topical Diagnostic and Therapeutic Transport

US20080045553A1—2004 Mar. 7 2008 Feb. 21 Phytotox Limited, Transdermal Administration of Phycotoxins

US20080057084A1—2006 Aug. 31 2008 Mar. 6 Allergan, Inc., Methods for selecting headache patients responsive to botulinum toxin therapy

US20080092910A1—2006 Oct. 18 2008 Apr. 24 Allergan, Inc., Apparatus and method for treating obesity using neurotoxins in conjunction with bariatric procedures

US20080113051A1—2006 Nov. 13 2008 May. 15 Allergan, Inc., Methods for alleviating tattoo pain

US20080193452A1—2004 Jul. 21 2008 Aug. 14 Cornell Research Foundation, Inc., Therapeutic Compounds Derived from Spider Venom and Their Method of Use

US20080199453A1—2007 Feb. 15 2008 Aug. 21 Gaylis Franklin D, Botulinum toxin compositions and methods

US20080213315A1—2000 Feb. 8 2008 Sep. 4 Allergan, Inc., Clostridial toxin pharmaceutical compositions

US20090104234A1—2007 Oct. 23 2009 Apr. 23 Allergan, Inc., Methods of treating chronic neurogenic inflammation using modified clostridial toxins

U.S. Pat. No. 7,537,773B1 1998 Aug. 25 2009 May 26 Botulinum Toxin Research Associates, Inc., Chemodenervating pharmaceutical as anti-inflammatory agent

US20090232850A1—2008 Mar. 13 2009 Sep. 17, Manack Aubrey N, Therapeutic treatments using botulinum neurotoxin

WO2009124046A1—2008 Apr. 3 2009 Oct. 8 Allergan, Inc., Suture line administration technique using botulinum toxins

US20100028385A1—2008 Aug. 4 2010 Feb. 4 Allergan, Inc., Treatment of excess cerumen secretion

U.S. Pat. No. 7,749,515B2 2005 Feb. 1 2010 Jul. 6 Allergan, Inc., Targeted delivery of botulinum toxin to the sphenopalatine ganglion

EP2204183A1 2002 Feb. 8 2010 Jul. 7 Allergan, Inc., Treatment of neuroblastoma

U.S. Pat. No. 7,758,872B1—2003 Feb. 7 2010 Jul. 20 Eric Finzi, Method of treating depression

U.S. Pat. No. 7,794,386B2 2006 Mar. 15 2010 Sep. 14 Allergan, Inc., Methods for facilitating weight loss

U.S. Pat. No. 7,811,584B2 2004 Jun. 30 2010 Oct. 12 Allergan, Inc., Multivalent clostridial toxins

US20100266638A1—2004 Feb. 26 2010 Aug. 21 Allergan, Inc., Headache treatment method

US20110054442A1—2003 Feb. 24 2011 Mar. 3 Ira Sanders, Cell Membrane Translocation of Regulated Snare Inhibitors, Compositions Therefor, and Methods for Treatment of Disease

WO2011038015A1 2009 Sep. 24 2011 Mar. 31 Allergan, Inc., Method of treating osteoporosis with a neurotoxin

US20110079535A1—2004 Jun. 30 2011 Apr. 7 Kimberly-Clark Worldwide, Inc., Sterilization Wrap with Additional Strength Sheet

US20110206752A1—2008 Jul. 24 2011 Aug. 25 Bcn Peptides, S.A., Compositions for the treatment of pain and/or inflammation

US20110206731A1—2003 Dec. 9 2011 Aug. 25 Allergan, Inc., Botulinum toxin therapy for skin disorders

WO2012051447A1 2010 Oct. 14 2012 Apr. 19 Allergan, Inc., Targeted delivery of targeted exocytosis modulators to the sphenopalatine ganglion for treatment of headache disorders

WO2012103415A1 2011 Jan. 28 2012 Aug. 2 Allergan, Inc., Dosage regimen for the treatment of multiple disorders with botulinum toxins

WO2012174123A1 2011 Jun. 13 2012 Dec. 20 Allergan, Inc., Treatment of psychological trauma

WO2013009625A1 2011 Jul. 8 2013 Jan. 17 Allergan, Inc., Method for treatment of autonomic nervous system disorders

WO2013010100A1 2011 Jul. 14 2013 Jan. 17 Allergan, Inc., Methods for treatment of incontinence associated with sexual activity

WO2013009584A1 2011 Jul. 8 2013 Jan. 17 Allergan, Inc., Method for treatment of esophageal spasm

WO2013013042A1 2011 Jul. 20 2013 Jan. 24 Allergan, Inc., Botulinum toxins for use in a method for treatment of adipose deposits

U.S. Pat. No. 8,420,106B1 2012 Mar, 12 2013 Apr. 16 William J. Binder, Extramuscular treatment of traumatic-induced migraine headache

U.S. Pat. No. 8,512,715B2 2008 Aug. 14 2013 Aug. 20 The Cleveland Clinic Foundation, Apparatus and method for treating a neuromuscular defect

WO2013137969A1 2012 Mar. 12 2013 Sep. 19 Binder William J, Treatment of migraine headaches with presynaptic neurotoxin

WO2013139965A2 2012 Mar. 22 2013 Sep. 26 Lipotec, S.A., Exopolysaccharide for the treatment and/or care of the skin, mucous membranes and/or nails

EP2649983A1 2012 Apr. 13 2013 Oct. 16 Lipotec, S.A., Compounds which inhibit neuronal exocytosis (II)

EP2649985A1 2012 Apr. 13 2013 Oct. 16 Lipotec, S.A., Compounds which inhibit neuronal exocytosis (III)

EP2649984A1 2012 Apr. 13 2013 Oct. 16 Lipotec, S.A., Compounds which inhibit neuronal exocytosis

WO2013153191A1 2012 Apr. 13 2013 Oct. 17 Lipotec, S.A., Compounds which inhibit neuronal exocytosis (ii)

WO2013180799A1 2012 May 30 2013 Dec. 5 President And Fellows Of Harvard College, Engineered botulinum neurotoxin

U.S. Pat. No. 8,697,090B2 2011 May 52014 Apr. 15 Allergan, Inc., Method of treating persistent genital arousal disorder with a neurotoxin

U.S. Pat. No. 8,841,110B2 2003 Sep. 25 2014 Sep. 23 Allergan, Inc., Animal product free system and process for purifying a botulinum toxin

WO2014147255A1 2013 Mar. 22 2014 Sep. 25 Lipotec S.A., Exopolysaccharide for the treatment and/or care of the skin, mucous membranes and/or nails

U.S. Pat. No. 8,940,308B2 2010 Mar. 30 2015 Jan. 27 Allergan, Inc., Methods for treating depression

US20150165003A1—2013 Dec. 12 2015 Jun. 18 Medy-Tox, Inc., Long lasting effect of new botulinum toxin formulations

U.S. Pat. No. 9,204,925B2 2008 Aug. 14 2015 Dec. 8 The Cleveland Clinic Foundation, Apparatus and method for treating a neuromuscular defect

U.S. Pat. No. 9,211,248B2 2004 Mar. 3 2015 Dec. 15 Revance Therapeutics, Inc., Compositions and methods for topical application and transdermal delivery of botulinum toxins

WO2016149092A1 2015 Mar. 13 2016 Sep. 22 Allergan, Inc., Improved injection paradigm for administration of botulinum toxins

WO2016154534A1 2015 Mar. 26 2016 Sep. 29 President And Fellows Of Harvard College, Engineered botulinum neurotoxin

WO2017214447A1 2016 Jun. 8 2017 Dec. 14 Children's Medical Center Corporation, Engineered botulinum neurotoxins

WO2018009903A2 2016 Jul. 8 2018 Jan. 11 Children's Medical Center Corporation, A novel botulinum neurotoxin and its derivatives

U.S. Pat. No. 9,901,627B2 2014 Jul. 18 2018 Feb. 27 Revance Therapeutics, Inc., Topical ocular preparation of botulinum toxin for use in ocular surface disease

WO2018039506A1 2016 Aug. 24 2018 Mar. 1 President And Fellows Of Harvard College, Engineered botulinum neurotoxin

U.S. Pat. No. 9,950,042B2 2008 Dec. 4 2018 Apr. 24 Revance Therapeutics, Inc., Extended length botulinum toxin formulation for human or mammalian use

GB9120306D0 1991 Sep. 24 1991 Nov. 6 Graham Herbert K, Method and compositions for the treatment of cerebral palsy

DE69435194D1—1993 Dec. 28 2009 Apr. 16 Allergan Sales Inc., Use of botulinum toxin type B for the manufacture of a medicament for reduction of pain due to muscle disease

EP1103267B1—1993 Dec. 28 2004 Sep. 15 Allergan, Inc., Botulinum toxins for treating pain associated with muscle disorders

U.S. Pat. No. 5,721,215A—1996 Mar. 2019 98 Feb. 24 Allergan, Injectable therapy for control of muscle spasms and pain related to muscle spasms

CA2319113A1 1998 Jan. 26 1999 Jul. 29 University Of Massachusetts, Biologically active hemagglutinin from type a clostridium botulinum and methods of use

WO2000062746A1—1999 Apr. 16 2000 Oct. 26 Marvin Schwartz, Method to reduce hair loss and stimulate hair regrowth

DE19925739A1 1999 Jun. 7 2000 Dec. 21 Biotecon Ges Fuer Biotechnologische Entwicklung & Consulting Mbh, Agent with a botulinum neurotoxin

US20020086036A1 2000 Dec. 5 2002 Jul. 4 Allergan Sales, Inc., Methods for treating hyperhidrosis

U.S. Pat. No. 7,255,865B2 2000 Dec. 5 2007 Aug. 14 Allergan, Inc., Methods of administering botulinum toxin

U.S. Pat. No. 6,767,544B2—2002 Apr. 1 2004 Jul. 27 Allergan, Inc., Methods for treating cardiovascular diseases with botulinum toxin

U.S. Pat. No. 8,617,572B2 2003 Oct. 29 2013 Dec. 31 Allergan, Inc., Botulinum toxin treatments of depression

DE102005058639A1 - 2005-12-072007-07-26 Armin Maurer, Treatment of Trichodynia and intumescent artery superficial temporal

U.S. Pat. No. 8,722,060B2—2012 May. 23 2014 May 13 William J. Binder, Method of treating vertigo

https://www.youtube.com/watch?v=TS23MBniX4E&t=14s.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a neurological-related disorder comprising applying a therapeutically effective amount of botulinum neurotoxin to nerve ganglia including sphenopalatine ganglia and/or other ganglia of the head and neck.

2. The method of claim 1, wherein the nerve ganglia is a parasympathetic nerve ganglia.

3. The method of claim 1, wherein the nerve ganglia is a sphenopalatine ganglia, a ciliary ganglia, a submandibular ganglia, superior cervical ganglia, trigeminal ganglia, stellate ganglia and/or an otic ganglia.

4. The method of claim 1, wherein the nerve ganglia is a sphenopalatine ganglia.

5. The method of claim 4, wherein the botulinum neurotoxin is applied to a pterygopalatine fossa.

6. The method of claim 1, wherein the botulinum neurotoxin is applied to the sphenopalatine ganglia.

7. The method of claim 6, wherein the botulinum neurotoxin is applied zygomatically, intranasally, through a hard palate technique, using a high tuberosity approach or combinations thereof

8. The method of claim 1, wherein the neurological disorder is chosen from the group consisting of cluster headache, migraine headache, trigeminal neuralgia, herpes zoster pain, facial head or neck pain from various sources, complex regional pain syndrome, nasal contact point headache and vasomotor rhinitis, TMJ disorders, headaches, migraines, myofascial pain and dysfunction, anxiety, panic attacks, problems associated with Autonomic Sympathetic Overload, dizziness, vertigo, tinnitus, vomiting and nausea related to chemotherapy or other disorders, high blood pressure, atrial fibrillation increased appetite and obesity, loss of libido in women and combinations thereof.

9. The method of claim 1, wherein the botulinum neurotoxin is chosen from the group consisting of botulinum neurotoxin type A, botulinum neurotoxin type B, botulinum neurotoxin type C, botulinum neurotoxin type D, botulinum neurotoxin type E, botulinum neurotoxin type F, botulinum neurotoxin type G, and combinations thereof.

10. The method of claim 1, wherein the botulinum neurotoxin is botulinum neurotoxin type A.

11. The method of claim 1, wherein the botulinum neurotoxin is botulinum neurotoxin type B.

12. The method of claim 11, wherein the botulinum neurotoxin type B is administered with epinephrine.

13. The method of claim 11, wherein the botulinum neurotoxin type B further comprises a basic solution.

14. The method of claim 1, wherein the amount of botulinum neurotoxin administered is between about 0.1 to about 1000 units.

15. The method of claim 1, wherein the amount of botulinum neurotoxin administered is between about 1 to about 1000 units.

16. The method of claim 1, wherein the amount of botulinum neurotoxin administered is between about 2 to about 50 units.

17. The method of claim 1, wherein the botulinum neurotoxin is administered over a period of time.

18. The method of claim 1, wherein the botulinum neurotoxin is administered over one minute.

19. The method of claim 1, wherein the volume of botulinum neurotoxin administered is between 0.1 to 10 cc.

20. The method of claim 1, wherein the botulinum neurotoxin is further administered locally to the skin.

21. The method of claim 1, wherein the botulinum neurotoxin is made from recombinant genetic methods.

22. The method of claim 1, wherein the botulinum toxin is isolated from Clostridia botulinum or Clostridia berratti.

23. A method of treating a pain syndrome comprising applying a therapeutically effective amount of botulinum neurotoxin to nerve ganglia including sphenopalatine ganglia and/or other ganglia of the head and neck.

24. The method of claim 23, wherein the nerve ganglia is a parasympathetic nerve ganglia.

25. The method of claim 23, wherein the nerve ganglia is a sphenopalatine ganglia, a ciliary ganglia, a submandibular ganglia, superior cervical ganglia, trigeminal ganglia, stellate ganglia and/or an otic ganglia.

26. The method of claim 23, wherein the nerve ganglia is a sphenopalatine ganglia.

27. The method of claim 26, wherein the botulinum neurotoxin is applied to a pterygopalatine fossa.

28. The method of claim 23, wherein the botulinum neurotoxin is applied to the sphenopalatine ganglia.

29. The method of claim 28, wherein the botulinum neurotoxin is applied zygomatically, intranasally, through a hard palate technique, using a high tuberosity approach or combinations thereof

30. The method of claim 23, wherein the pain syndrome results from a disorder chosen from the group consisting of migraine headaches, including migraine headaches with aura, migraine headaches without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches; chronic cluster headaches; cluster variants; chronic paroxysmal hemicrania; hemicrania continua; post-traumatic headache; post-traumatic neck pain; post-herpetic neuralgia involving the head or face; pain from spine fracture secondary to osteoporosis; arthritis pain in the spine, headache related to cerebrovascular disease and stroke; headache due to vascular disorder; reflex sympathetic dystrophy, cervicalgia; glossodynia, carotidynia; cricoidynia; otalgia due to middle ear lesion; gastric pain; sciatica; maxillary neuralgia; laryngeal pain, myalgia of neck muscles; trigeminal neuralgia; post-lumbar puncture headache; low cerebro-spinal fluid pressure headache; temporomandibular joint disorder; atypical facial pain; ciliary neuralgia; paratrigeminal neuralgia; petrosal neuralgia; Eagle's syndrome; idiopathic intracranial hypertension; orofacial pain; myofascial pain syndrome involving the head, neck, and shoulder; chronic migraneous neuralgia, cervical headache; paratrigeminal paralysis; sphenopalatine ganglion neuralgia; carotidynia; Vidian neuralgia; and causalgia; trigeminal neuralgia, herpes zoster; back pain and sciatica and combinations thereof.

31. The method of claim 23, wherein the botulinum neurotoxin is chosen from the group consisting of botulinum neurotoxin type A, botulinum neurotoxin type B, botulinum neurotoxin type C, botulinum neurotoxin type D, botulinum neurotoxin type E, botulinum neurotoxin type F, botulinum neurotoxin type G, and combinations thereof.

32. The method of claim 23, wherein the botulinum neurotoxin is botulinum neurotoxin type A.

33. The method of claim 23, wherein the botulinum neurotoxin is botulinum neurotoxin type B.

34. The method of claim 33, wherein the botulinum neurotoxin type B is administered with epinephrine.

35. The method of claim 33, wherein the botulinum neurotoxin type B further comprises a basic solution.

36. The method of claim 23, wherein the amount of botulinum neurotoxin administered is between about 0.1 to about 1000 units.

37. The method of claim 23, wherein the amount of botulinum neurotoxin administered is between about 1 to about 1000 units.

38. The method of claim 23, wherein the amount of botulinum neurotoxin administered is between about 2 to about 50 units.

39. The method of claim 23, wherein the botulinum neurotoxin is administered over a period of time.

40. The method of claim 23, wherein the botulinum neurotoxin is administered over one minute.

41. The method of claim 23, wherein the volume of botulinum neurotoxin administered is between 0.1 to 10 cc.

42. The method of claim 23, wherein the botulinum neurotoxin is further administered locally to the skin.

43. The method of claim 23, wherein the botulinum neurotoxin is made from recombinant genetic methods.

44. The method of claim 23, wherein the botulinum toxin is isolated from Clostridia botulinum or Clostridia berratti.
